(19) United States
(12) Patent Application Publication
(10) Pub. No.: US 2026/0314932 A1
(43) Pub. Date: Oct. 15, 2026

(54) EYE IMAGING DEVICE AND EYE IMAGING METHOD

(71) Applicant: NIKON CORPORATION, Tokyo (JP)

(72) Inventors: Hiroshi KASAI, Kawasaki-shi (JP); Masahiro MIZUTA, Yokohama-shi (JP); Jiro INOUE, Yokohama-shi (JP); Mitsuhiro YANARI, Tokyo (JP)

(21) Appl. No.: 19/464,796

(22) Filed: Jan. 30, 2026

Related U.S. Application Data
(63) Continuation of application No. PCT/JP2024/026675, filed on Jul. 25, 2024.

(30) Foreign Application Priority Data
Jul. 31, 2023 (JP) 2023-124985

(51) Int. Cl. A61B 3/15; A61B 3/00; A61B 3/117

(52) U.S. Cl. CPC A61B 3/15; A61B 3/0041; A61B 3/117

(57) ABSTRACT

An eye imaging device for imaging an eye that includes a retroreflection unit that is configured to retroreflect light from the eye, a beam splitter that is arranged on a light path of light from the eye to the retroreflection unit, and an acquisition unit that is configured to acquire, from among the light from the eye, light via the retroreflection unit and the beam splitter.

### CROSS-REFERENCE TO RELATED APPLICATION

This application is a continuation application of International Application No. PCT/JP2024/026675, filed Jul. 25, 2024, the disclosure of which is incorporated herein by reference in its entirety. Further, this application claims priority from Japanese Patent Application No. 2023-124985, filed Jul. 31, 2023, the disclosure of which is incorporated herein by reference in its entirety.

### TECHNICAL FIELD

Technology disclosed herein relates to an eye imaging device, and an eye imaging method.

### BACKGROUND ART

In order to ascertain states by observing and/or identify person states, various types of eye imaging device have been implemented that are able to image the inside and outside of an eye of a subject who is the person targeted for state acquisition, and the periphery thereof. For example, an eye imaging device is known that is able to observe the eye of a subject, such as an object eye of a patient or the like for the purpose of eye diagnostics and eye surgical treatment in ophthalmology. For example, technology related to a device including an optical system that forms a real image of a fundus of an object eye is known (see, Japanese Patent Application Publication 2000-504251).

### SUMMARY OF INVENTION

A first aspect of technology disclosed herein is
an eye imaging device for imaging an eye, including:
a retroreflection unit that is configured to retroreflect light from the eye;
a beam splitter that is arranged on a light path of light from the eye to the retroreflection unit; and
an acquisition unit that is configured to acquire, from among the light from the eye, light via the retroreflection unit and the beam splitter.

A second aspect of technology disclosed herein is
an eye imaging device for imaging an eye, including:
a retroreflection unit that is configured to retroreflect light from the eye;
a beam splitter that is configured to reflect, and guide to the retroreflection unit, light from the eye, and to transmit light that has been retroreflected at the retroreflection unit; and
an acquisition unit that is configured to acquire, from among the light from the eye, light that has been retroreflected by the retroreflection unit and transmitted by the beam splitter.

A third aspect of technology disclosed herein is
an eye imaging device for imaging an eye, including:
a retroreflection unit that is configured to retroreflect incident light;
a beam splitter that is configured to transmit, and guide to the retroreflection unit, light from the eye, and to reflect, and guide to an acquisition unit, light that has been retroreflected by the retroreflection unit; and
the acquisition unit, which is configured to acquire, from among the light from the eye, light that has been retroreflected by the retroreflection unit and reflected by the beam splitter.

### BRIEF DESCRIPTION OF DRAWINGS

FIG. 1 is a block diagram illustrating an example of a configuration of an eye imaging device according to a first exemplary embodiment.

FIG. 2 is a schematically illustrating relating to illuminating light onto an object eye in an eye imaging device according to the first exemplary embodiment.

FIG. 3 is a diagram illustrating an example of a configuration to reduce stray light in an eye imaging device according to the first exemplary embodiment.

FIG. 4 is a block diagram illustrating an example of a configuration of an eye imaging device according to a second exemplary embodiment.

FIG. 5 is a block diagram illustrating an example of a configuration of an eye imaging device according to a third exemplary embodiment.

FIG. 6 is a block diagram illustrating an example of a configuration of an eye imaging device according to a fourth exemplary embodiment.

FIG. 7 is a block diagram illustrating an example of a configuration of an eye imaging device according to a fifth exemplary embodiment.

FIG. 8 is a block diagram illustrating an example of a configuration of an eye imaging device according to a sixth exemplary embodiment.

FIG. 9 is a block diagram illustrating an example of a configuration of an eye imaging device according to a seventh exemplary embodiment.

FIG. 10 is a flowchart illustrating an example of a flow of processing in an eye imaging device.

FIG. 11 is a block diagram illustrating an example of a configuration of an eye imaging device according to a modified example of the fourth exemplary embodiment.

### DESCRIPTION OF EMBODIMENTS

Detailed explanation follows regarding an exemplary embodiment to implement technology disclosed herein, with reference to the drawings. Note that the same reference numerals are appended across the drawings for configuration elements and processing having the same action or function, and sometimes duplicate explanation thereof is omitted as appropriate. Moreover, sometimes explanation is omitted for configuration not directly related to technology disclosed herein, or known configuration. Moreover, sometimes description will be omitted for configuration not directly related to the technology disclosed herein and for known configuration. The dimensions and proportions in the drawings may be exaggerated for ease of explanation, and sometimes differ from actual proportions. Furthermore, each of the drawings is merely illustrated schematically to such a degree as to enable sufficient understanding of the technology disclosed herein. The technology disclosed herein is accordingly not limited to the examples illustrated.

An eye imaging device according to technology disclosed herein is applicable to any device that images an eye of a subject who is a target person (hereinafter called the object eye) and a region including the object eye. For example, a device that acquires images related to eyes, such as fundus images or the like, is applicable. In the present exemplary embodiment, in order to simplify the following description, as an example of an eye imaging device including a function to acquire images, description follows of application to an eye imaging device such as an ophthalmic device or the like for observing an object eye of a patient or the like, for example the fundus that is a posterior eye portion or the object eye periphery for the purpose of eye diagnostics and eye surgical treatment in ophthalmology, or an ophthalmic device or the like for an observer such as a doctor to observe an anterior eye portion, for example. Note that the technology disclosed herein is obviously not limited to being an eye imaging device of an ophthalmic device or the like in ophthalmology.

### First Exemplary Embodiment

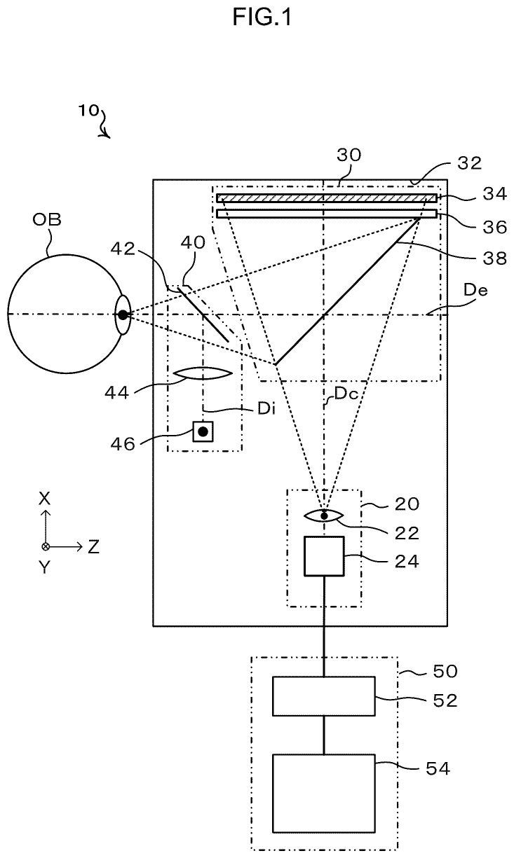

FIG. 1 illustrates an example of a configuration of an eye imaging device 10 according to the present exemplary embodiment. As illustrated in FIG. 1, the eye imaging device 10 includes an image acquisition unit 20, an ocular optical unit 30, and an illumination unit 40.

In the following description, with the eye imaging device 10 installed on a horizontal plane parallel to the ground, an eye width direction of a subject is denoted the “Y direction”, and a direction perpendicular to the horizontal plane that the eye imaging device 10 is installed on is denoted the “X direction”, and a depth direction of object eye OB (namely, the front-rear direction of an axis centered on the gaze of the object eye OB) is denoted the “Z direction”. Note that the X direction and the Y direction may be reversed. Moreover, the X direction and the Y direction may be directions with respect to the configuration of the eye imaging device 10. For example, for the X direction and the Y direction, a horizontal direction when the eye imaging device 10 is installed at a specific position (for example, on a horizontal plane) may be the X direction, and a vertical direction may be the Y direction, or the reverse.

The image acquisition unit 20 is a functional unit for acquiring images related to the object eye OB, and acquires images of the object eye OB using light from the object eye OB. The ocular optical unit 30 is a functional unit for guiding light from the object eye OB to the image acquisition unit 20, and includes a retroreflection element 34 that retroreflects light from the object eye OB, and guides the light from the object eye OB to the image acquisition unit 20 via the retroreflection element 34. Note that retroreflection is reflection in which an angle of an incident direction of incident light is maintained as is, and reflection is in the opposite direction to the incident direction. The illumination unit 40 is a functional unit that illuminates the object eye OB, and illuminates the object eye OB such that a light path for illuminating the object eye OB is not via the retroreflection element 34. The eye imaging device 10 accordingly illuminates the object eye OB with the illumination light of the illumination unit 40 not via the retroreflection element 34, with the light from the object eye OB being guided by the ocular optical unit 30 to the image acquisition unit 20 via the retroreflection element 34. The retroreflection element 34 is an example of a retroreflection unit of the present disclosure.

The ocular optical unit 30 includes the retroreflection element 34, a wave plate 36, and a beam splitter 38. Note that preferably a λ/4 plate is applied as the wave plate 36. Moreover, in cases in which a λ/4 plate is applied as the wave plate 36, the λ/4 plate is preferably one that functions in a wide wavelength band from visible light to near infrared light. Obviously a λ/4 plate that is a wave plate that functions only across part of the wavelength band from visible light to near infrared light is also suitable. The beam splitter 38 is arranged on the light path of light from the object eye OB to reach the retroreflection element 34. The ocular optical unit 30 functions as an optical system to guide light from the object eye OB to the image acquisition unit 20 via the retroreflection element 34. Note that the ocular optical unit 30 is housed in a casing 32.

The ocular optical unit 30 deflects (i.e. reflects) the light from the object eye OB toward the retroreflection element 34 with the beam splitter 38, retroreflects this light with the retroreflection element 34, and guides the retroreflected light to the image acquisition unit 20 through the beam splitter 38. As an example of the retroreflection element 34, an element may be applied that reflects the light in the opposite direction to the direction of incident light to the element using plural orthogonal reflection faces. The retroreflection element 34 is an optical element that includes reflecting incident light in a perpendicular direction toward a reflection site back in the opposite direction, and reflects incident light having an incident direction at a freely selected incident angle to reflection site in the opposite direction to the incident direction while maintaining the angle of the incident direction. In the present exemplary embodiment, a polarizing beam splitter is preferably applied as the beam splitter 38. A polarizing beam splitter is an optical element to split incident light into reflected light and transmitted light, with light of one polarized light component being reflected from the incident light out of a polarized light component from S polarized light and a polarized light component from P polarized light, and with light of other polarized components being transmitted. Namely, light from the object eye OB propagating in the Z direction is reflected by the beam splitter 38 toward the retroreflection element 34. On the other hand, light that has been retroreflected at the retroreflection element 34 has the polarized light components of incident light and reflected light changed by the wave plate 36, and passes through the beam splitter 38 toward the image acquisition unit 20. The retroreflection element 34, the beam splitter 38, and the image acquisition unit 20 are arranged in a row along a second direction intersecting with a first direction connecting the object eye OB and the beam splitter 38.

In the ocular optical unit 30, the beam splitter 38 is arranged at a position of intersection between an axis De that is a central axis of the object eye OB in a direction along the visual axis (gaze direction), and an axis Dc that is a direction along a central axis of an imaging lens 22, described later, such that light from the object eye OB is reflected substantially at right angles. The central axis of light reflected at right angles aligns with the axis Dc that is the direction along the central axis of the imaging lens 22, described later. The wave plate 36 and the retroreflection element 34 are arranged in this sequence on the reflection side of the beam splitter 38.

Note that although in the example illustrated in FIG. 1 an example is given of a case in which the light from the object eye OB is reflected substantially at a right angle by the beam splitter 38, the technology disclosed herein is not limited to the light from the object eye OB being reflected substantially at a right angle by the beam splitter 38. Namely, as long as the light from the object eye OB is reflected by the beam splitter 38 and deflected toward the retroreflection element 34, for example, the beam splitter 38 may be arranged so as to reflect light at a more acute angle than a right angle, or may be arranged so as to reflect light at a more obtuse angle than a right angle.

Moreover, although in the present exemplary embodiment an example has been described of a case in which a polarizing beam splitter is applied as the beam splitter 38, the technology disclosed herein is not limited to application of a polarizing beam splitter. For example, the beam splitter 38 may employ a reflection mirror such as a half mirror or the like that reflects part of the incident light, and may employ a wavelength selective mirror such as a dichroic mirror that reflects light of a predetermined wavelength.

In the ocular optical unit 30 including the wave plate 36 as described above, the wave plate 36 may be omitted. The wave plate 36 functions effectively in cases in which a polarizing beam splitter is employed for the beam splitter 38. However, in cases in which a reflection mirror or the like, such as a half mirror or the like, is employed as the beam splitter 38 to simply split light, there is no need for the optical function of the wave plate 36, and so the wave plate 36 may be omitted.

Specifically, by using a polarizing beam splitter as the beam splitter 38, the wave plate 36 is able to reflect and transmit the incident light according to polarized components, enabling light to be split efficiently. Moreover, a wave plate (for example, a ¼ wave plate) is preferably arranged between the beam splitter 38 and the retroreflection element 34 when a polarizing beam splitter is employed as the beam splitter 38.

Description follows regarding cases in which a wave plate is not arranged between the beam splitter 38 and the retroreflection element 34. Light from the eye is split by the beam splitter 38, which is a polarizing beam splitter, into a polarized light component transmitted by the beam splitter 38 and a polarized light component reflected by the beam splitter 38, with the polarized light component reflected by the beam splitter 38 directed toward the retroreflection element 34. This light is retroreflected by the retroreflection element 34 toward the beam splitter 38. The light that has been reflected at the retroreflection element 34 progresses toward the beam splitter 38 without its polarization state being changed. This means that this light would not be transmitted through the beam splitter 38, and would instead be reflected at the beam splitter 38, making it difficult to get this light to reach the image acquisition unit 20. However, by arranging the wave plate between the beam splitter 38 and the retroreflection element 34, the light reflected by the beam splitter 38 is able to be guided to the image acquisition unit 20. Namely, light of the polarized light component reflected at the beam splitter 38 passes in sequence via the wave plate 36, the retroreflection element 34 (reflector), and the wave plate 36, and due to passing via the wave plate 36 a second time becomes light of a polarized light component transmitted by the beam splitter 38. This means that the light passing via the wave plate 36 and the retroreflection element 34 from out of the light reflected by the beam splitter 38 becomes light that is able to be transmitted by the beam splitter 38, and accordingly reaches the image acquisition unit 20. The beam splitter 38 transmits all of the light that has passed via the wave plate 36 and the retroreflection element 34, and so the image acquisition unit 20 is able to efficiently acquire light from the eye reflected by the beam splitter 38. This means that the collection efficiency of light from the eye is able to be improved by using the beam splitter 38, which is a polarizing beam splitter, and the wave plate 36.

The casing 32 is attached to a non-illustrated pedestal, and is formed so as to have no contact with the subject with the object eye OB when imaging the object eye OB. Due to the ocular optical unit 30 being formed so as to have no contact with the subject, an uncomfortable feeling of the subject arising through contact of the subject with the eye imaging device 10 is suppressed.

The ocular optical unit 30 is arranged at a position separated by a predetermined specific distance from the object eye OB. A lens optical system is employed in an ordinary eye imaging device for imaging the retina. In an eye imaging device using a lens optical system, often a distance between the device and the object eye is set to several tens of millimeters when acquiring a fundus image, with the subject experiencing uncomfortable pressing during imaging. The eye imaging device 10 according to the present exemplary embodiment is able to secure distance of several hundreds of millimeters or greater between the object eye OB and the ocular optical unit 30. Namely, the distance from the object eye OB to the beam splitter 38 nearest in the field of view of the object eye OB is formed at a separation of at least 100 mm or greater. The eye imaging device 10 is accordingly able to reduce an uncomfortable pressing sensation experienced by the subject during imaging compared to with an ordinary eye imaging device.

The image acquisition unit 20 acquires an image of the object eye OB, and so in the present exemplary embodiment, as an example, is configured so as to function as an imaging unit for imaging the object eye OB. The image acquisition unit 20 includes an imaging lens 22 and an image sensor 24. The image acquisition unit 20 serves as an example of an acquisition unit of the present disclosure, and acquires an image of the object eye OB by condensing light from the object eye OB onto the image sensor 24 using the imaging lens 22. Note that the image acquisition unit 20 may be configured so as, in addition to the object eye OB, to acquire an image of a periphery of the object eye OB.

In the image acquisition unit 20, the imaging lens 22 functions as an imaging optical system. The imaging lens 22 forming the imaging optical system may employ an optical lens configured by a single lens, or by a lens group of a complex of plural lenses.

The imaging lens 22 has a predetermined angle of view, with a position of the pupil of the imaging lens 22 and the position of the pupil of the object eye OB arranged conjugate to each other such that the angle of the light converged at the object eye OB by the retroreflection element 34 is the same angle. The beam splitter 38, the retroreflection element 34, and the imaging lens 22 thereby function as an optical system to condense the light from the object eye OB (signal light) onto the image sensor 24.

In the image acquisition unit 20, the imaging lens 22 and the image sensor 24 are arranged in this sequence at the transmission side of the beam splitter 38 for light from the object eye OB. A central axis of light from the object eye OB aligns with an image acquisition direction of the image acquisition unit 20, i.e. aligns with the axis Dc in the direction along the central axis (i.e. the optical axis) of the imaging lens 22.

Note that the image acquisition unit 20 may employ an imaging device that includes an imaging lens capable of imaging an imaging subject and, for example, an imaging device of a mobile terminal having an imaging function (for example, a so-called smartphone) may be employed therefor.

The illumination unit 40 includes a beam splitter 42, an illumination lens 44, and a light source 46. The illumination unit 40 is an example of an illumination unit of technology disclosed herein, and functions as an illumination-light optical system for illuminating the object eye OB with light from the light source 46. The illumination unit 40 is arranged between the object eye OB and the ocular optical unit 30 so as to illuminate the object eye OB with the light from the light source 46. Specifically, the beam splitter 42, the illumination lens 44, and the light source 46 are arranged in this sequence from the object eye side in the space between the object eye OB, and the beam splitter 38 of the ocular optical unit 30. The beam splitter 42, the illumination lens 44, and the light source 46 are arranged in the illumination unit 40 such that a position of the light source 46 and a position of the pupil of the object eye OB are conjugate to each other.

The beam splitter 42 of the illumination unit 40 is arranged on the axis De direction along the visual axis (gaze direction) of the object eye OB so as to reflect the light from the light source 46 arriving via the illumination lens 44, by reflection toward the object eye OB substantially at right angles.

Note that although the example illustrated in FIG. 1 is an example of a case in which the light from the light source 46 is reflected substantially at right angles by the beam splitter 42, technology disclosed herein is not limited to technology in which light is reflected substantially at right angles by the beam splitter 42. Namely, as long as light from the light source 46 is reflected by the beam splitter 42 and deflected toward the object eye OB, for example, the beam splitter 42 may be arranged so as to reflect light at a more acute angle than a right angle, or may be arranged so as to reflect light at a more obtuse angle than a right angle.

The light source 46 may employ a light source that emits light in a range from the visible light wavelength region to the near infrared wavelength region, for example, light in a range from 450 nm to 1100 nm. Examples of the light source 46 include LED light sources and laser light sources. A configuration may be adopted that employs a white light source, and an optical filter that transmits light from the visible light wavelength region to the near infrared wavelength region. Moreover, a configuration may be adopted in which an optical fiber is employed so as to propagate light.

Note that although in the present exemplary embodiment a description has been given of a case in which a light source including a light generator is employed to emit light as the light source 46, there is no limitation thereto. For example, the light source 46 may lack a light generator, and be configured so as to include a light introduction function that introduces light from an external light source into the device.

The illumination unit 40 illuminates the light emitted from the light source 46 onto the object eye OB by using the illumination lens 44 to condense light at a specific position on the object eye OB. This means that the illumination lens 44 functions as a condensing optical system to condense light at the specific position on the object eye OB. The illumination unit 40 is able to illuminate the fundus of the object eye OB. Detailed description is given later regarding the position for condensing light onto the object eye OB illuminated by the illumination unit 40. Note that the illumination lens 44 is an example of a condensing optical system of technology disclosed herein.

The illumination lens 44 that forms the condensing optical system may employ an optical lens configured by a single lens, or by a lens group of a complex of plural lenses. An example of an optical lens is a Fresnel lens.

In the illumination unit 40, the light source 46 is arranged on an axis Di along the illumination direction, namely a direction along a central axis (i.e. the optical axis) of the illumination lens 44, and the beam splitter 42 is arranged at a position of intersection between the axis De and the axis Di. The beam splitter 42 reflects the light of the light source 46 via the illumination lens 44 so as to be illuminated onto the object eye OB. The illumination unit 40 is accordingly able to illuminate the object eye OB without the illumination-light light path for illuminating the object eye OB being via the retroreflection element 34.

Namely, the illumination unit 40 is arranged in the space between the object eye OB and the ocular optical unit 30, and so the illumination light is not guided to the image acquisition unit 20 directly via the retroreflection element 34. This means that there is no effect from scattered light, generated by illumination light of the illumination unit 40 being incident to the retroreflection element 34, being acquired as stray light by the image acquisition unit 20, so as to generate a ghost image in the acquired object eye OB image.

Note that FIG. 1 illustrates an example in which the illumination unit 40 is arranged between the object eye OB and the ocular optical unit 30 at the interior of the casing 32, however technology disclosed herein is not limited thereto. Namely, as long as the illumination unit 40 is able to illuminate the object eye OB such that the illumination-light light path is not via the retroreflection element 34, configuration of at least part of the illumination unit 40 may be arranged inside the ocular optical unit 30. For example, the light source 46, or at least part of the illumination lens 44 or the light source 46, may be arranged inside the ocular optical unit 30. Moreover, the entirety of the illumination unit 40 may be arranged inside the ocular optical unit 30.

The eye imaging device 10 described above may include a display unit 50 for displaying an image related to the object eye OB acquired by the image acquisition unit 20. The display unit 50 enables an image related to the object eye OB to be presented to an observer or the like. The display unit 50 is an example of a display unit of the present disclosure.

As illustrated in the example of FIG. 1, the display unit 50 is a functional unit for displaying images related to the object eye OB acquired by the image acquisition unit 20, and is connected to the image sensor 24 of the image acquisition unit 20. The display unit 50 includes an image sensor controller 52 and a display 54. The image sensor 24 of the image acquisition unit 20 described above is electronic equipment that converts images related to the object eye OB into a picture signal, and the image sensor controller 52 of the display unit 50 is electronic equipment that converts the picture signal into a display signal and outputs the display signal. The image sensor controller 52 is connected to the display 54, typified by a liquid crystal monitor or the like, and outputs the display signal to the display 54. An image imaged by the image sensor 24 is accordingly displayed on the display 54. The observer is able to set an observation position on the object eye OB or at the periphery of the object eye OB by manipulating the positions of the ocular optical unit 30 and the image acquisition unit 20 while looking at the images being displayed on the display 54. The display 54 may be arranged to as to be visible to the subject. The subject may adjust the observable position on the object eye OB or the periphery of the object eye OB by moving with respect to the eye imaging device 10 while looking at the images being displayed on the display 54.

The image sensor controller 52 may, for example, be implemented by a configuration including a computer that includes a non-illustrated CPU, RAM, ROM, and input-output interface, which are connected together through a bus so as to be able to exchange various commands and data. An image processing program may be included on the non-illustrated ROM for executing image processing to enable an observer to appropriately perceive captured images. Operation as the image sensor controller 52 to perform image processing enabling the observer to appropriately perceive captured images is implemented by this image processing program being read from the non-illustrated ROM, expanded in the RAM, and the image processing program that has been expanded in the RAM being executed by the CPU.

Note that the image acquisition unit 20 and the display unit 50 may exchange information by wired communication using a wired connection, or may exchange information by wireless communication using a wireless connection.

Moreover, the information for exchange between the image acquisition unit 20 and the display unit 50 is not limited to being a display signal. For example, operation information of the image acquisition unit 20 may be contained therein. Examples of such operation information include information indicating at least one instrument operating condition, such as an optical magnification of the image acquisition unit 20, an electronic magnitude of the image sensor 24, or the like.

Next, description follows regarding illumination light for illuminating onto the object eye OB by the illumination unit 40.

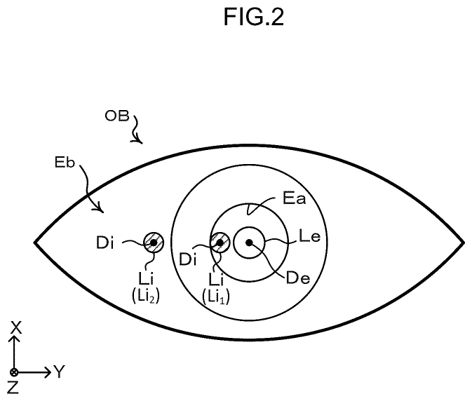

FIG. 2 is a schematic diagram related to a position of the illumination light condensed on the object eye OB.

Firstly, in the eye imaging device 10, this position is set inside a pupil Ea such that an imaging light path Le of the imaging optical system configured by the imaging lens 22 of the image acquisition unit 20 passes through the pupil Ea. A fundus image of the object eye OB is accordingly able to be acquired with the image acquisition unit 20. For example, the ocular optical unit 30 and the image acquisition unit 20 are adjusted such that the axis of the imaging light path Le formed by light propagating in a direction along the axis Dc via the retroreflection element 34 and the beam splitter 38 aligns with the axis De in the direction along the visual axis of the object eye OB (see FIG. 1). Note that in FIG. 2 the position of the imaging light path Le at the pupil Ea is illustrated inside the pupil Ea.

The illumination unit 40 illuminates the object eye OB with the illumination light from the light source 46. The illumination lens 44 condenses the illumination light from the light source 46 onto the object eye OB. Note that in FIG. 2, for the illumination light that the illumination unit 40 illuminates the object eye OB with, a position of the illumination-light light path of the illumination light at the surface of the object eye OB and the pupil Ea is indicated as illumination-light light path Li, which is a light beam area. The illumination unit 40 may be configured such that the illumination-light light path Li of the illumination light onto the object eye OB (for example, a condensing position of condensed illumination light) is configured so as to illuminate one or other position on the object eye OB, however preferably configuration is such that the illumination-light light path Li does not overlap with the position of the imaging light path Le of the imaging optical system at the pupil Ea. Configuring the illumination unit 40 such that the position of the illumination-light light path Li of the illumination light at the pupil Ea does not overlap with the position of the imaging light path Le, enables illumination light scattered by the pupil Ea to be prevented from mixing as stray light into the imaging light path Le.

Specific examples of the illumination unit 40 for illuminating the object eye OB will now be described. The illumination unit 40 may be configured so as to satisfy any of the following examples. A first example is an example in which the illumination unit 40 is configured so as to produce an illumination-light light path Li (Li₁) to illuminate the interior of the pupil Ea of the object eye OB. The illumination-light light path Li (Li₁) may employ the illumination position of the condensing position by the illumination unit 40, or the like. In the first example, the illumination unit 40 is configured so as to position the illumination-light light path Li (Li₁) inside the pupil Ea such that illumination light onto the object eye OB passes through the pupil Ea. Moreover, the illumination unit 40 of the first example is configured so as to position the illumination-light light path Li (Li₁) at a different position to the position of the imaging light path Le inside the pupil Ea and not overlapping with the imaging light path Le. This means that the illumination unit 40 illuminates the object eye OB with illumination light that has passed through the pupil Ea and has progressed along a light path different to the imaging light path Le. The illumination light from the light source 46 is accordingly able to illuminate the fundus of the object eye OB through the pupil Ea. Moreover, due to the position of the illumination-light light path Li and the position of the imaging light path Le in the pupil Ea being separate non-overlapping light paths, illumination light scattered by the pupil Ea does not mix as stray light into the imaging light path Le. The illumination unit 40 illuminating the illumination light so as to pass through the pupil Ea functions effectively for acquiring fundus images to observe the fundus. The illumination unit 40 for illuminating the pupil is an example of a condensing optical unit of the present disclosure.

A second example is an example in which the illumination unit 40 is configured so as to produce an illumination-light light path Li (Li₂) to illuminate the sclera of the object eye OB. In the second example, the illumination unit 40 is configured so as to position the illumination-light light path Li (Li₂) of the illumination light on the sclera of the object eye OB (a location on the so-called white of the eye). The light irradiated onto the sclera generates multiple scattering at the sclera, enabling the fundus to be illuminated by the multiple scattering light. The sclera of the object eye OB is positioned away from the pupil Ea, and so the illumination-light light path Li (Li₂) and the imaging light path Le are separated. In such cases, the light irradiated onto the sclera preferably does not overlap with the imaging light path Le at the pupil Ea. For example, the illumination unit 40 may be configured so as to restrict the size of the light beam of illumination light, and may be configured so as to control the illumination position, such that part of the illumination light on the sclera does not mix into the imaging light path Le (described in detail later). Note that although the sclera absorbs light in a near infrared wavelength region, this absorption is small compared to that of visible light. This means that a light source for emitting light in the near infrared wavelength region is preferably employed as the light source 46. Employing light in the near infrared wavelength region is in order to be able to reduce the absorption of light by multiple scattering at the sclera. The illumination unit 40 is accordingly able to illuminate the fundus of the object eye OB by illuminating irradiation light onto the sclera. The illumination unit 40 of the second example functions effectively for acquiring fundus images for observing the fundus by illuminating irradiation light onto the sclera. The illumination unit 40 for illuminating the sclera is an example of a condensing optical unit of the present disclosure.

A third example is an example in which the illumination unit 40 is configured so as to illuminate an anterior eye portion of the object eye OB. In the third example, the illumination unit 40 is configured such that the illumination-light light path Li of the illumination light is positioned at a predetermined location for observing the anterior eye portion of the object eye OB. When observing the anterior eye portion of the object eye OB, the size for observing the anterior eye portion can be adjusted by setting a predetermined size for a size of the location for condensing light (condensed light spot). This thereby enables the anterior eye portion of the object eye OB to be illuminated by the illumination unit 40. The illumination of the anterior eye portion of the object eye OB by the illumination unit 40 of the third example functions effectively for acquiring anterior eye portion images for observing the anterior eye portion of the object eye OB.

Note that in a third example, the imaging lens 22 of the image acquisition unit 20 can be omitted as long as the anterior eye portion of the object eye OB can be observed. Namely, in an eye imaging device omitting the imaging lens 22, the object eye OB and the image sensor 24 may be set at conjugate positions. Namely, the anterior eye portion of the object eye OB may be arranged such that an image of the anterior eye portion is formed on the image sensor 24 by the retroreflection element 34. The image sensor 24 of the third example illustrates an example of an image sensor of the present disclosure.

Adjustment of the illumination-light light path Li of the illumination light onto the object eye OB described above, namely of the illumination position of the condensing position or the like, may be adjustment by adjusting an optical system including an ocular optical unit, an illumination unit, and an image acquisition unit. Note that this adjustment may be performed based on information related to a position of an object eye as acquired by a position observation unit 70, described later, by adjustment performed by a position adjustment unit, also described later.

Moreover, when acquiring an image of the anterior eye portion of the object eye OB using the imaging lens 22 and the image sensor 24, focus adjustment may be performed in the image acquisition unit 20 to make the focal point align with the anterior eye portion of the object eye OB, by adjusting the focal length of the imaging lens 22, by adjusting the separation between the imaging lens 22 and the image sensor 24, or the like. Note that in the eye imaging device 10 using the retroreflection element 34, in cases in which focus adjustment is performed according to the position of the object eye OB when imaging an anterior eye portion, this focus adjustment is in the opposite direction to the adjustment direction of an ordinary lens system with respect to the movement direction of the object eye OB. When the object eye OB has been moved in a direction (Z direction) so as to approach the ocular optical unit 30, when via an optical lens, the focal position would be moved in a direction away from the ocular optical unit 30. On the other hand, in cases in which the retroreflection element 34 is employed, when the object eye OB has been moved in the direction (Z direction) so as to approach the ocular optical unit 30, the focal position is moved in a direction to approach the ocular optical unit 30. This means that focus adjustment when imaging an anterior eye portion in the eye imaging device 10 employing the retroreflection element 34 results in focus adjustment in the opposite direction to in an eye imaging device employing an optical lens.

Next, description follows regarding operation of the eye imaging device 10 according to the present exemplary embodiment.

Firstly, description follows regarding a case in which the image acquisition unit 20 acquires a fundus image of the object eye OB as an image related to the object eye OB.

The eye imaging device 10 acquires a fundus image of the object eye OB in the image acquisition unit 20 through the ocular optical unit 30 using light from the eye (see FIG. 1). Namely, light from the fundus of the object eye OB is reflected at the beam splitter 38 of the ocular optical unit 30 and, after passing through the wave plate 36, is retroreflected by the retroreflection element 34. The retroreflected light is condensed by the imaging lens of the image acquisition unit 20 and formed as an image on the image sensor 24. The image acquisition unit 20 accordingly acquires a fundus image of the object eye OB.

On the other hand, illumination light is illuminated onto the fundus of the object eye OB from the illumination unit 40. The illumination light illuminated onto the fundus of the object eye OB is light illuminating the object eye OB at a different position to the imaging light path Le of the imaging optical system (see FIG. 2). Moreover, the illumination unit 40 is arranged in the space between the object eye OB and the ocular optical unit 30, and the illumination-light light path Li is set outside the imaging light path Le of the imaging optical system, and so the illumination light does not reach the retroreflection element 34 (FIG. 1). This means that scattering light, generated by the illumination light of the illumination unit 40 being incident to the retroreflection element 34, is not acquired as stray light by the image acquisition unit 20, and does not generate a ghost image in the fundus image of the object eye OB.

Although in the above description is of a case in which a fundus image of the object eye OB is acquired by the image acquisition unit 20, technology disclosed herein is not limited thereto. A description will now be given regarding a case in which an anterior eye portion image of the object eye OB is acquired by the image acquisition unit 20 as an image related to the object eye OB.

In the eye imaging device 10, an anterior eye portion image of the object eye OB is acquired in the image acquisition unit 20 using the light from the anterior eye portion via the ocular optical unit 30. Namely, the light from the anterior eye portion of the object eye OB is reflected by the beam splitter 38 of the ocular optical unit 30, passes through the wave plate 36, and is retroreflected by the retroreflection element 34. The retroreflected light is condensed by an imaging lens of the image acquisition unit 20 and is formed as an image on the image sensor 24. Images of an anterior eye portion the object eye OB are accordingly acquired by the image acquisition unit 20.

The anterior eye portion of the object eye OB is illuminated with the illumination light from the illumination unit 40. The illumination light illuminated onto the anterior eye portion of the object eye OB illuminates the object eye OB at a different position to the imaging light path Le of the imaging optical system. Moreover, the illumination unit 40 is arranged in a space between the object eye OB and the ocular optical unit 30, and the illumination-light light path Li is set outside the imaging light path Le of the imaging optical system and so the illumination light does not reach the retroreflection element 34. This means that scattering light, generated by the illumination light of the illumination unit 40 being incident to the retroreflection element 34, is not acquired as stray light by the image acquisition unit 20, and does not generate a ghost image in the anterior eye portion images of the object eye OB.

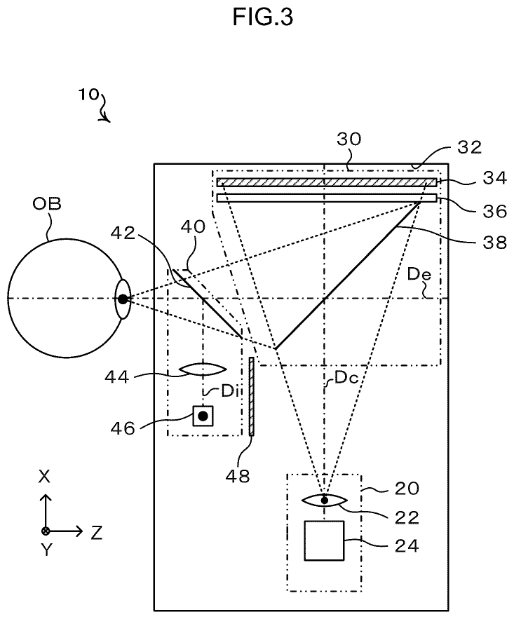

Note that in the present exemplary embodiment, as described above, the illumination unit 40 is arranged in the space between the object eye OB and the ocular optical unit 30 such that scattering light generated by the illumination light of the illumination unit 40 being incident to the retroreflection element 34 does not become stray light. In order to reduce the light emitted from the illumination unit 40 reaching the ocular optical unit 30 and the image acquisition unit 20 (stray light), as illustrated in FIG. 3, a light blocking element 48 serving as a light reduction unit is preferably provided in the space between the illumination unit 40 and the ocular optical unit 30. For example, the light blocking element 48 is arranged so as to surround at least part of a light beam area passed by light for illuminating the object eye OB, reducing the amount of light from out of the light for illuminating the object eye OB that leaks out in directions other than toward the object eye OB.

As described above, in the present exemplary embodiment, the illumination unit 40 for illuminating the object eye OB is arranged in the space between the object eye OB and the ocular optical unit 30 for guiding light to the image acquisition unit 20. The illumination light accordingly does not reach the image acquisition unit 20 directly via the retroreflection element 34. This means that scattering light, generated by the illumination light being incident to the retroreflection element 34, is not acquired as stray light by the image acquisition unit 20, and does not generate a ghost image the images of the object eye OB.

### Second Exemplary Embodiment

Next, description follows regarding a second exemplary embodiment. Note that the second exemplary embodiment is configured similarly to the first exemplary embodiment, and so the same reference numerals are appended to the same parts thereof, and detailed explanation thereof will be omitted. Moreover, the second exemplary embodiment will be described by way of parts differing to the first exemplary embodiment.

In the first exemplary embodiment, as the illumination unit 40, a case has been described in which the beam splitter 42 is arranged on the axis De such that light from the light source 46 is reflected toward the object eye OB substantially at right angles. Technology disclosed herein is not limited to light from the light source 46 being reflected toward the object eye OB by the beam splitter 42. The second exemplary embodiment will be described for the case of an eye imaging device that lacks the beam splitter 42.

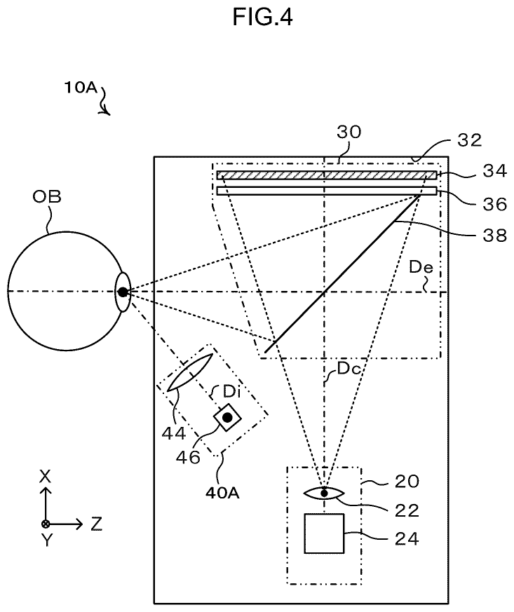

FIG. 4 illustrates an example of a configuration of an eye imaging device 10A according to the present exemplary embodiment.

As illustrated in FIG. 4, the eye imaging device 10A includes an illumination unit 40A instead of the illumination unit 40 illustrated in FIG. 1. The illumination unit 40A includes an illumination lens 44 and a light source 46, but lacks the beam splitter 42 illustrated in FIG. 1. Note that the display unit 50 illustrated in FIG. 1 is omitted from illustration in FIG. 4.

The illumination unit 40A functions as an illumination-light optical system for illuminating light of the light source 46 directly onto the object eye OB via the illumination lens 44. The illumination unit 40A is, similarly to the illumination unit 40, arranged between the object eye OB and the ocular optical unit 30. Specifically, the illumination lens 44 and the light source 46 are arranged in the space between the object eye OB and the beam splitter 38 of the ocular optical unit 30, in this sequence from the object eye side. In the illumination unit 40A, the illumination lens 44 and the light source 46 are arranged such that the position of the light source 46 and the position of the pupil of the object eye OB are conjugate to each other.

Moreover, in the illumination unit 40, the light source 46 is arranged on an axis Di of an illumination direction, that is, along a central axis (i.e., an optical axis) of the illumination lens 44, and is arranged such that a pupil of the object eye OB is located on the axis Di. Namely, in the illumination unit 40, the axis Di, which is the optical axis of the light source 46 via the illumination lens 44, is arranged so as to have an inclination of a specific angle with respect to the axis De. The specific angle, which is the angle between the axis Di of the illumination light and the axis De, may be set to a freely selected angle such that the object eye OB and the ocular optical unit 30 do not interfere with the illumination unit 40A. The illumination unit 40A is accordingly able to illuminate the object eye OB without the illumination-light light path for illuminating the object eye OB being via the retroreflection element 34.

Note that the illumination unit 40A is preferably arranged so as to illuminate the sclera of the object eye OB as described above. Illuminating the sclera of the object eye OB enables light to be scattered effectively at the sclera. This means that, even when the object eye OB is irradiated with off-axis illumination light along an axis Di that is inclined by a specific angle with respect to an axis De, the fundus can be illuminated by light scattered at the sclera, thereby obtaining a quantity of light sufficient for fundus imaging.

Moreover, although FIG. 4 illustrates an example in which the illumination unit 40A is arranged between the object eye OB and the ocular optical unit 30 at the inside of the casing 32, technology disclosed herein is not limited thereto. Namely, as long as the illumination unit 40A is able to illuminate the object eye OB such that the illumination-light light path is not via the retroreflection element 34, configuration of at least part of the illumination unit 40A may be arranged outside the ocular optical unit 30. For example, at least one out of the illumination lens 44 or the light source 46 may be arranged outside the ocular optical unit 30.

As described above, in the present exemplary embodiment, the illumination unit 40A for illuminating the object eye OB is configured without being equipped with a beam splitter, thereby enabling the number of components of the device for illuminating the object eye OB to be reduced.

### Third Exemplary Embodiment

Next, description follows regarding a third exemplary embodiment. Note that the third exemplary embodiment is configured similarly to the first exemplary embodiment, and so the same reference numerals are appended to the same parts thereof, and detailed explanation thereof will be omitted. Moreover, the third exemplary embodiment will be described by way of parts differing to the first exemplary embodiment.

In the first exemplary embodiment, the illumination unit 40 was described for a case in which the beam splitter 42 is arranged on the axis De such that light from the light source 46 is reflected substantially at right angles toward the object eye OB. The third exemplary embodiment will be described for an eye imaging device in a case in which the light source 46 is arranged on an extension line of the axis De.

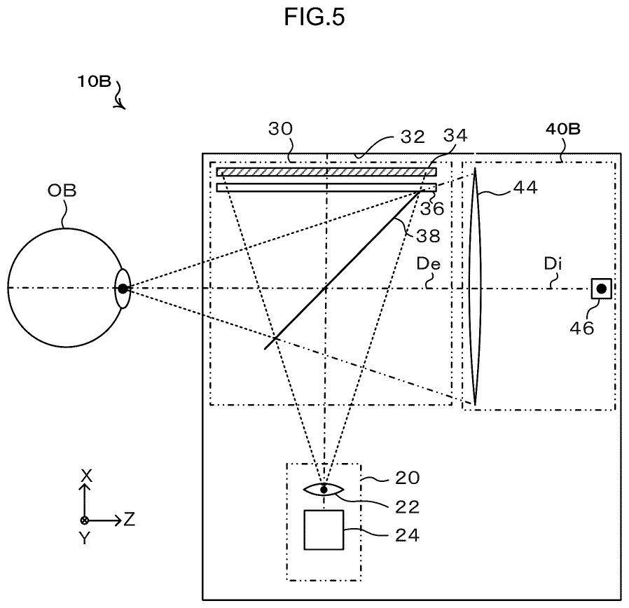

FIG. 5 illustrates an example of a configuration of an eye imaging device 10B according to the present exemplary embodiment.

As illustrated in FIG. 5, the eye imaging device 10B includes an illumination unit 40B instead of the illumination unit 40 illustrated in FIG. 1. The illumination unit 40B lacks the beam splitter 42 illustrated in FIG. 1, and includes an illumination lens 44B and a light source 46 arranged so as to form an axis Di along an extension line of the axis De.

Specifically, in the illumination unit 40B, the illumination lens 44B and the light source 46 are arranged in this sequence from the object eye side so as to form the illumination light axis Di in a direction along the axis De at the opposite side of the ocular optical unit 30 to the object eye OB side. Moreover, in the illumination unit 40B, the illumination lens 44B and the light source 46 are arranged such that the position of the light source 46 and the position of the pupil of the object eye OB are conjugate to each other. The illumination lens 44B includes a lens having an aperture lens capable of illuminating illumination light that completely encompasses an angle of view for acquiring images with the image acquisition unit 20 (the angle of view of the imaging optical system). Note that although the aperture of the illumination lens 44B preferably encompasses the angle of view to acquire an image with the image acquisition unit 20, there is no limitation thereto. For example, the aperture of the illumination lens 44B may be set such that the object eye OB is irradiated at an angle of view smaller than the angle of view for acquiring images with the image acquisition unit 20.

The illumination unit 40B illuminates the object eye OB by condensing light that has been emitted from the light source 46 to a specific position on the object eye OB using the illumination lens 44. Specifically, the illumination unit 40B condenses the light from the light source 46 inside a pupil Ea of the object eye OB with the illumination lens 44B. The light emitted from the illumination lens 44B, which is the light from the light source 46, is condensed inside the pupil Ea of the object eye OB via the beam splitter 38, but is not directed toward the retroreflection element 34. This means that the illumination unit 40B is able to illuminate the object eye OB without the illumination-light light path for illuminating the object eye OB being via the retroreflection element 34. Accordingly, the illumination light from the illumination unit 40B does not reach the image acquisition unit 20 directly via the retroreflection element 34. This means that scattering light generated by the illumination light of the illumination unit 40 being incident to the retroreflection element 34 is not acquired as stray light by the image acquisition unit 20.

As described above, in the present exemplary embodiment, the illumination light path arising from the illumination unit 40B illuminating the object eye OB is set to a different light path to the imaging light path of light from the object eye OB, the illumination light does not reach the image acquisition unit 20 via the retroreflection element 34, and the illumination light does not appear as a ghost image in the image of the object eye OB.

Moreover, in the present exemplary embodiment, the illumination unit 40B that illuminates the object eye OB is arranged at a position on the opposite side of the ocular optical unit 30 to the object eye OB side, namely, at a position separated from the object eye OB. This means that an uncomfortable pressing sensation experienced by the subject can be reduced compared to cases in which at least part of the illumination unit for illuminating the object eye OB is provided in the space between the ocular optical unit 30 (for example, the beam splitter 38) and the object eye OB.

### Fourth Exemplary Embodiment

Next, description follows regarding a fourth exemplary embodiment. Note that the fourth exemplary embodiment is a configuration similar to that of the first exemplary embodiment and the third exemplary embodiment, and so the same reference numerals are appended to the same parts thereof, and detailed explanation thereof will be omitted. Moreover, description follows of parts differing from the exemplary embodiments described above.

The third exemplary embodiment was described for an eye imaging device in a case in which, as the illumination unit 40, the light source 46 was arranged on the extension line of the axis De. The fourth exemplary embodiment is a modified example to guide the illumination light in a direction along the axis De. Moreover, the fourth exemplary embodiment employs a retroreflection element also for use with illumination light.

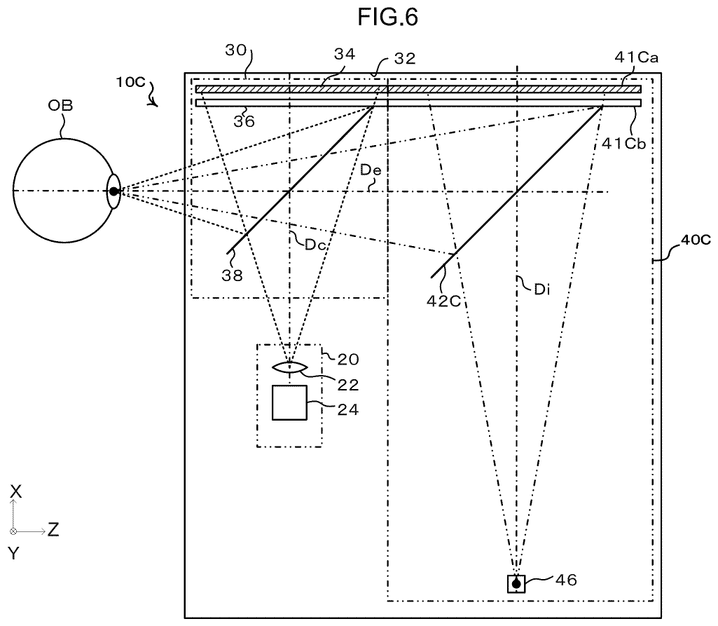

FIG. 6 illustrates an example of a configuration of an eye imaging device 10C according to the present exemplary embodiment.

As illustrated in FIG. 6, the eye imaging device 10C includes an illumination unit 40C instead of the illumination unit 40B illustrated in FIG. 5. The illumination unit 40C is a configuration similar to that of the ocular optical unit 30 described above, and includes a retroreflection element 41Ca, a wave plate 41Cb, and a beam splitter 42C.

Specifically, in the illumination unit 40C, the retroreflection element 41Ca, the wave plate 41Cb, the beam splitter 42C, and a light source 46 are arranged on the opposite side of the ocular optical unit 30 to the object eye OB side. Moreover, in the illumination unit 40C, the retroreflection element 41Ca, the wave plate 41Cb, the beam splitter 42C, and the light source 46 are arranged such that the position of the light source 46 and the position of the pupil of the object eye OB are conjugate to each other.

Note that the retroreflection element 41Ca and the wave plate 41Cb of the illumination unit 40C may be arranged as an extension of the retroreflection element 34 and the wave plate 36 of the ocular optical unit 30 described above, or may be arranged as separate individual configuration to the retroreflection element 34 of the ocular optical unit 30, and the wave plate 36. Configuring as an extension of the retroreflection element 34, and the wave plate 36 enables commonality of adjustment of the retroreflection element 34 and of the wave plate 36. Moreover, separate configuration to the retroreflection element 34 and the wave plate 36 enables configuration at respective sizes according to the light beam for acquiring images, and the light beam for illumination, and enables optical members to be appropriately utilized without waste.

The illumination unit 40C functions as an optical system to illuminate the object eye OB by guiding the light from the light source 46 to the object eye OB via the retroreflection element 41Ca and the beam splitter 42C. Namely, in the illumination unit 40C, the light from the light source passes through the beam splitter 42C, and progresses to the retroreflection element 41Ca. The light that has been retroreflected at the retroreflection element 41Ca is deflected (i.e. reflected) toward the object eye OB by the beam splitter 42C, and is condensed at the object eye OB. The beam splitter 42C preferably employs a polarizing beam splitter similar to the beam splitter 38.

In the illumination unit 40C, the beam splitter 42C is arranged at a position of intersection between the axis De of the direction along the visual axis of the object eye OB, and the axis Di of the direction along the illumination direction that is the central axis (i.e. the optical axis) of the light source 46. The beam splitter 42C transmits light from the light source 46, and is arranged such that light retroreflected by the retroreflection element 41Ca of the transmitted light is reflected substantially at right angles toward the object eye OB. The central axis of the light reflected at right angles aligns with the axis De. The wave plate 41Cb and the retroreflection element 41Ca are arranged in sequence along the progression direction of light of the light source 46 transmitted through the beam splitter 42C.

Note that the example illustrated in FIG. 6 illustrates an example of a case in which the light retroreflected by the retroreflection element 41Ca is reflected substantially at right angles by the beam splitter 42C, however technology disclosed herein is not limited to the light being reflected substantially at right angles by the beam splitter 42C. Namely, as long as the light retroreflected by the retroreflection element 41Ca heads toward the object eye OB, for example, the beam splitter 42C may be arranged such that the light is reflected at an angle more acute than a right angle, and may be arranged such that the light is reflected at an angle more obtuse than a right angle.

Moreover, although in the example illustrated in FIG. 6, an example is given in which the optical system formed by the ocular optical unit 30 including the beam splitter 38 and the image acquisition unit 20 is arranged from the object eye OB side, and the illumination unit 40C including the beam splitter 42C is arranged at the downstream side thereof, technology disclosed herein is not limited thereto. For example, the illumination unit 40C including the beam splitter 42C may be arranged at an upstream position on the object eye OB side, and the optical system formed by the ocular optical unit 30 including the beam splitter 38 and the image acquisition unit 20 may be arranged at the downstream side thereof.

Moreover, although FIG. 6 illustrates the ocular optical unit 30 including the wave plate 36 and the beam splitter 38 as described above, and the illumination unit 40C including the wave plate 41Cb, the wave plate 36 and the wave plate 41Cb may be omitted. Note that the wave plate 36 as described above functions efficiently in cases in which a polarizing beam splitter is employed as the beam splitter 38, with the image acquisition unit able to effectively condense light from the eye. Moreover, the illumination unit 40C is preferably configured including a wave plate 41Cb. By employing a polarizing beam splitter in the beam splitter 42C similarly to the beam splitter 38, light from the light source 46 is able to be reflected or transmitted according to polarized components, enabling light to be guided efficiently to the object eye OB. Namely, in cases in which a polarizing beam splitter is employed as the beam splitter 42C, arranging a wave plate between the beam splitter 42C and the retroreflection element 41Ca enables light transmitted and reflected at the beam splitter 42C to be guided to the object eye OB. Specifically, the light via the wave plate 41Cb and the retroreflection element 41Ca of light transmitted by the beam splitter 42C is reflected by the beam splitter 42C, guided to the ocular optical unit 30, and reaches the object eye OB. In cases in which a polarizing beam splitter is employed as the beam splitter 38 of the ocular optical unit 30, the beam splitter 38 is preferably configured so as to transmit the polarized component of light propagated from the beam splitter 42C. However, in cases in which a beam splitter that simply splits the light from the beam splitter 42C is employed, such as a half mirror, sampling mirror, or the like, the optical function of the wave plate 41Cb is not needed, and so the wave plate 41Cb may be omitted.

Moreover, although in the present exemplary embodiment a case has been described in which a polarizing beam splitter is employed as the beam splitter 42C, technology disclosed herein is not limited to employing a polarizing beam splitter. For example, a reflection mirror to reflect part of the incident light may be employed, such as a half mirror or the like, or a wavelength selective mirror may be employed, such as a dichroic mirror that reflects light of a predetermined wavelength.

This means that in the present exemplary embodiment, the illumination unit 40C illuminates the object eye OB by condensing the light emitted from the light source 46 at a specific position of the object eye OB using the retroreflection element 41Ca for illumination. Namely, the illumination unit 40C condenses the light from the light source 46 inside the pupil Ea of the object eye OB using the retroreflection element 41Ca for illumination. The light heading toward the object eye of light from the light source 46 directly progresses from the beam splitter 42C toward the object eye OB, and not toward the retroreflection element 34 for image acquisition. This means that the illumination unit 40C is able to illuminate the object eye OB without the illumination-light light path for illuminating the object eye OB being via the retroreflection element 34. This thereby enables a configuration in which the illumination light from the illumination unit 40C does not reach the image acquisition unit 20 directly via the retroreflection element 34. This means that scattering light generated by the illumination light of the illumination unit 40C being incident to the retroreflection element 34 is not acquired as stray light by the image acquisition unit 20.

As described above, the retroreflection element 41Ca is employed for illumination in the present exemplary embodiment, enabling an illumination lens 44 to be omitted. Moreover, the illumination-light light path from the illumination unit 40C for illuminating the object eye OB is a different light path to the imaging light path of light from the object eye OB, and so the illumination light does not reach the image acquisition unit 20 via the retroreflection element 34, and the illumination light does not appear as a ghost image in the image of the object eye OB.

### Modified Example

Next, description follows regarding a modified example of the fourth exemplary embodiment. This modified example is a modified example including an illumination unit for illuminating using the retroreflection element, and is configured similarly to the above exemplary embodiments, and so the same reference numerals are appended to the same parts thereof, and detailed explanation thereof will be omitted.

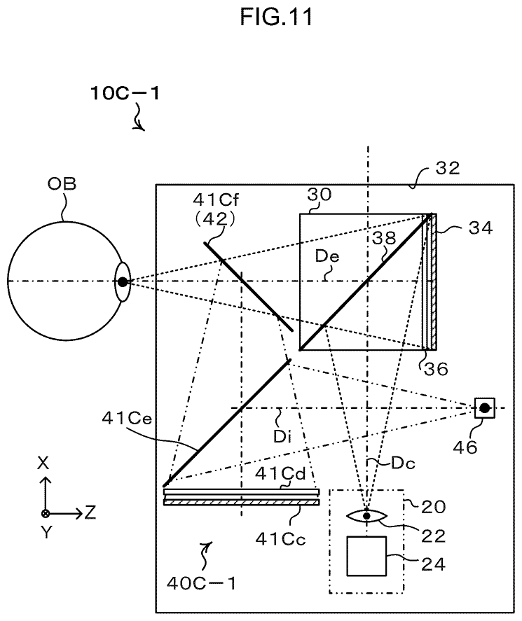

FIG. 11 illustrates an example of a configuration of an eye imaging device 10C-1 according to the modified example.

As illustrated in FIG. 11, the eye imaging device 10C-1 includes an illumination unit 40C-1 instead of the illumination unit 40C (FIG. 6). The illumination unit 40C-1 includes a a light source 46, a retroreflection element 41Cc, a wave plate 41Cd, a beam splitter 41Ce, and a beam splitter 41Cf. The illumination unit 40C-1 uses the light source 46, the retroreflection element 41Cc, the wave plate 41Cd, the beam splitter 41Ce, and the beam splitter 41Cf to illuminate the object eye OB. The beam splitter 41Cf may be configured similarly to the beam splitter 42 described above.

The illumination unit 40C-1 deflects (i.e. reflects) the light from the light source 46 toward the retroreflection element 41Cc at the beam splitter 41Ce, and retroreflects this light at the retroreflection element 41Cc. Moreover, in the illumination unit 40C-1, light that has been retroreflected at the retroreflection element 41Cc is transmitted through the beam splitter 41Ce, is deflected (i.e. reflected) by the beam splitter 41Cf toward the object eye OB, and is condensed at the object eye OB. Specifically, in the illumination unit 40C-1, the light source 46, the beam splitter 41Ce, the wave plate 41Cd, the retroreflection element 41Cc, and the beam splitter 41Cf are arranged in this sequence of illumination light toward the object eye OB. In the illumination unit 40C-1, similarly to the illumination unit 40C (FIG. 6), the retroreflection element 41Cc, the wave plate 41Cd, the beam splitters 41Ce, 41Cf, and the light source 46 are arranged such that a position of the light source 46 and a position of the pupil of the object eye OB are conjugate to each other. At least part of the illumination unit 40C-1 is arranged in a space between the object eye OB and the ocular optical unit 30. The example illustrated in FIG. 11 is an example in which the beam splitter 41Ce is arranged in the space between the object eye OB and the ocular optical unit 30.

Namely, the beam splitter 41Ce is arranged at an emission side of light of the light source 46. The retroreflection element 41Cc is arranged at the reflection side of light from the light source 46 of the beam splitter 41Ce, and the wave plate 41Cd is arranged between the beam splitter 41Ce and the retroreflection element 41Cc. The beam splitter 41Cf is arranged at a retroreflected side of light of the retroreflection element 41Cc. This means that light from the light source 46 is guided by the beam splitter 41Ce to the retroreflection element 41Cc. The light that has been retroreflected by the retroreflection element 41Cc is transmitted through the beam splitter 41Ce, and guided by the beam splitter 41Cf along the visual axis (axis De) of the object eye OB.

In the illumination unit 40C-1, the visual axis (axis De) of the object eye OB, and the optical axis (axis Di) of the light source 46 are at different positions to each other, and the optical axis (axis Dc) of the image acquisition unit 20 and the optical axis (axis Di) of the light source 46 intersect with each other. Moreover, an axis of light retroreflected by the retroreflection element 41Cc of light from the light source 46, intersects with the visual axis (axis De) of the object eye OB, and is at a different position to the optical axis (axis Dc) of the image acquisition unit 20. This means that light from the light source 46 does not mix in to the light path of light from the object eye OB to the image acquisition unit 20 (namely, the light path configured with the axis De and the axis Dc).

Note that although the example illustrated in FIG. 11 illustrates an example in which the beam splitters 41Ce, 41Cf reflect light substantially at right angles, similarly to as described above, technology disclosed herein is not limited to the light being reflected substantially at right angles at the beam splitters 41Ce, 41Cf. For example, reflection may be performed at a predetermined acute or obtuse angle.

The beam splitter 41Cf described above preferably employs a polarizing beam splitter in order to guide light from the eye efficiently to the object eye OB to the image acquisition unit 20. In cases in which a polarizing beam splitter is employed as the beam splitter 41Cf, the illumination unit 40C-1 is preferably configured including a wave plate 41Cd, however the wave plate 41Cd may be omitted. The illumination unit 40C-1 enables light to be guided according to polarized components due to employing a polarizing beam splitter as the beam splitter 41Cf and arranging the wave plate 41Cd between the beam splitter 41Cf and the retroreflection element 41Cc. Namely, the illumination unit 40C-1 is able to efficiently guide light to the object eye OB and the image acquisition unit 20.

Specifically, light via the wave plate 41Cd and the retroreflection element 41Cc of light from the light source 46 is reflected by the beam splitter 41Cf and guided to the object eye OB. Light from the object eye OB is transmitted through the beam splitter 41Cf and guided to the image acquisition unit 20 via the ocular optical unit 30. This means that the beam splitter 41Cf is preferably configured so as to reflect a polarized component of light from the retroreflection element 41Cc, and to transmit a polarized component from the eye. On the other hand, when a beam splitter that simply splits the light is employed for the beam splitter 41Cf, such as a half mirror, sampling mirror, or the like, the optical function of the wave plate 41Cd is not required, and so the wave plate 41Cd may be omitted. Note that as described above, the beam splitter 41Cf is not limited to employing a polarizing beam splitter, and may employ a reflection mirror, and may employ a wavelength selective mirror.

Thus in the modified example, in the illumination unit 40C-1, similarly to the illumination unit 40C, the object eye OB is illuminated by the light emitted from the light source 46 being condensed onto a specific position of the object eye OB by the retroreflection element 41Cc for illumination. The light toward the object eye of light from the light source 46 is guided by the beam splitter 41Cf to the object eye OB, and so does not head toward the retroreflection element 34 for image acquisition. The illumination unit 40C-1 accordingly enables the object eye OB to be illuminated with the illumination-light light path for illuminating the object eye OB that is not via the retroreflection element 34. This means that the illumination light from the illumination unit 40C-1 does not reach the image acquisition unit 20 directly via the retroreflection element 34. This means that scattering light generated by the illumination light of the illumination unit 40C-1 being incident to the retroreflection element 34 is not acquired as stray light by the image acquisition unit 20.

As described above, the retroreflection element 41Cc for illumination is employed in the modified example, enabling the object eye OB to be illuminated without using a lens. Moreover, the illumination light path of the illumination unit 40C-1 is a different light path to the imaging light path of light from the object eye OB, and so the illumination light does not reach the image acquisition unit 20 via the retroreflection element 34, and the illumination light does not become stray light and appear as a ghost image in the image of the object eye OB.

### Fifth Exemplary Embodiment

Next, description follows regarding a fifth exemplary embodiment. Note that the fifth exemplary embodiment is configured similarly to the exemplary embodiments described above, and so the same reference numerals are appended to the same parts thereof, and detailed explanation thereof will be omitted. Moreover, description follows of parts differing from the exemplary embodiments described above.

In the first exemplary embodiment, a case has been described in which, in the ocular optical unit 30, the light from the object eye OB is reflected by the beam splitter 38, and an image of the object eye OB is acquired by the image acquisition unit 20 using retroreflected light resulting from this reflected light being retroreflected by the retroreflection element 34. The fifth exemplary embodiment is an eye imaging device in which, in the ocular optical unit 30, the functions of light reflection and light transmission of the beam splitter 38 and the retroreflection element 34 have been interchanged.

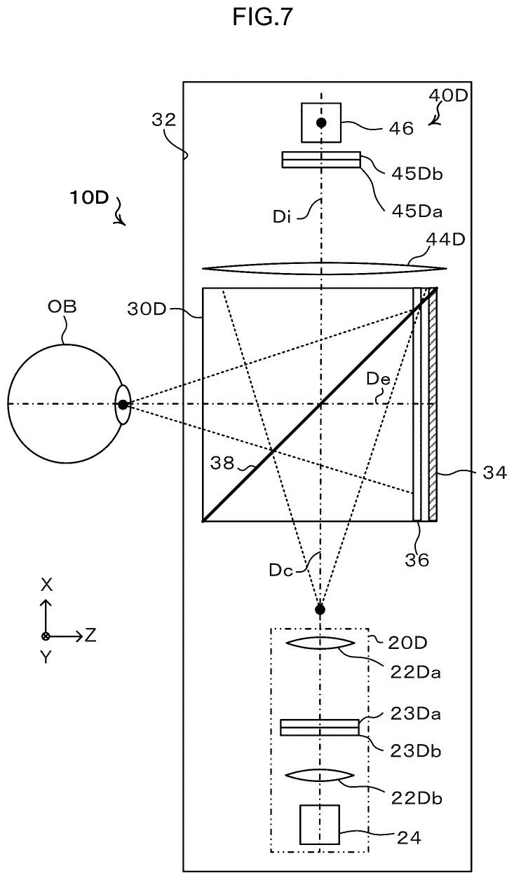

FIG. 7 illustrates an example of a configuration of an eye imaging device 10D according to the present exemplary embodiment.

As illustrated in FIG. 7, the eye imaging device 10D includes an image acquisition unit 20D, an ocular optical unit 30D, and an illumination unit 40D of configuration similar to each section of the eye imaging device 10B illustrated in FIG. 5. In the ocular optical unit 30D, the retroreflection element 34 is arranged on the axis De in a direction along the visual axis of the object eye OB via the beam splitter 38 and the wave plate 36. Namely, the beam splitter 38 is arranged so as to transmit light from the object eye OB. At the transmission side of the beam splitter 38 for light from the object eye OB, the wave plate 36 and the retroreflection element 34 are arranged in this sequence from the object eye OB side. Moreover, the beam splitter 38 reflects light that has been retroreflected by the retroreflection element 34. The image acquisition unit 20D is arranged at the reflection side of the beam splitter 38 for light that has been retroreflected by the retroreflection element 34. The beam splitter 38 preferably employs a polarizing beam splitter.

In the present exemplary embodiment, the image acquisition unit 20D includes a relay optical system to relay a position (conjugate position) where light from the object eye OB that has been retroreflected by the retroreflection element 34 is condensed, to the image sensor 24. Specifically, a first imaging lens 22Da and a second imaging lens 22Db are included. Note that when a polarizing beam splitter is employed as the beam splitter 38 in the image acquisition unit 20D, in order to improve an extinction ratio of light from the eye, namely, in order to reduce the incidence of stray light having a different polarized component to the light from the eye incident via the beam splitter 38 and the retroreflection element 34, the image acquisition unit 20D includes a polarizing element 23Da and a wave plate 23Db. The polarizing element 23Da and the wave plate 23Db may be omitted. Note that the wave plate 36 configured as described above functions effectively when a polarizing beam splitter is employed as the beam splitter 38, enabling light from the object eye OB to be guided to the image acquisition unit 20D while also enabling stray light to be excluded, and the wave plate 36 may be omitted when a half mirror or the like is employed as the beam splitter 38.

An illumination unit 40D is arranged on the opposite side of the ocular optical unit 30D to the image acquisition unit 20D side. Namely, the illumination lens and the light source 46 are arranged on the axis Dc of a direction along an image acquisition direction of the image acquisition unit 20 in this sequence from the ocular optical unit 30D side. Note that, in order to control polarized components of the illumination light, namely such that the image acquisition unit 20 does not acquire illumination light transmitted by the beam splitter 38 as stray light, the illumination unit 40D may include a polarizing element 45Da and a wave plate 45Db. The polarizing element 45Da and the wave plate 45Db may be omitted.

The polarizing element 23Da and the wave plate 23Db, and also the polarizing element 45Da and the wave plate 45Db, are examples of a polarization control unit of the present disclosure.

In the present exemplary embodiment, the light emitted from the light source 46 by the illumination unit 40D is reflected by the beam splitter 38, and the object eye OB is illuminated. The light toward the object eye OB of the light from the light source 46 is reflected by the beam splitter 38 toward the object eye OB, and so does not head toward the retroreflection element 34. The illumination unit 40D is accordingly able to illuminate the object eye OB without the illumination-light light path for illuminating the object eye OB being via the retroreflection element 34. The illumination light from the illumination unit 40D accordingly does not reach the image acquisition unit 20D directly via the retroreflection element 34. This means that scattering light generated by the illumination light of the illumination unit 40D being incident to the retroreflection element 34 is not acquired as stray light by the image acquisition unit 20D.

As described above, even in a case in which there has been an exchange of functions related to light reflection and light transmission of the beam splitter 38 and the retroreflection element 34 in the ocular optical unit 30, as in the present exemplary embodiment, the illumination light does not reach the image acquisition unit 20 via the retroreflection element, and illumination light does not become stray light and appear as a ghost image in images of the object eye OB.

Note that although in the example illustrated in FIG. 7 a case has been described in which the beam splitter 38 of the ocular optical unit 30D is utilized commonly for the image acquisition unit 20D and the illumination unit 40D, obviously these may be configured independently. For example, the beam splitter 38 of the ocular optical unit 30D may be employed in the image acquisition unit 20D, and the beam splitter 42 of the illumination unit 40 such as illustrated in FIG. 1 may be arranged as a separate beam splitter, and light from the light source 46 illuminated onto the object eye OB.

### Sixth Exemplary Embodiment

Next, description follows regarding a sixth exemplary embodiment. Note that the sixth exemplary embodiment is configured similarly to the exemplary embodiments described above, and so the same reference numerals are appended to the same parts thereof, and detailed explanation thereof will be omitted. Moreover, description follows of parts differing from the exemplary embodiments described above.

In the exemplary embodiments described above, a case has been described in which images of the object eye OB are acquired using the light from the object eye OB. The sixth exemplary embodiment is an eye imaging device employing both eyes of the left eye and the right eye as the object eyes.

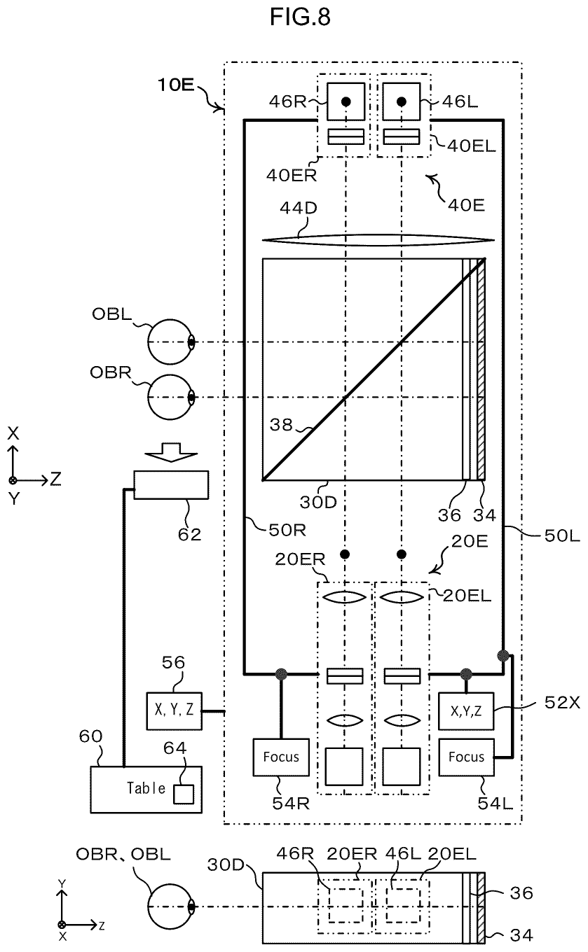

FIG. 8 illustrates an example of a configuration of an eye imaging device 10E according to the present exemplary embodiment.

As illustrated in FIG. 8, the eye imaging device 10E includes each part of a configuration similar to the eye imaging device 10D illustrated in FIG. 7 for each of the left eye and the right eye. An ocular optical unit employs the ocular optical unit 30D (FIG. 7) commonly for both eyes, i.e. the left eye and the right eye. Namely, the eye imaging device 10E includes the ocular optical unit 30D configured similarly to the eye imaging device 10D (FIG. 7). In the present exemplary embodiment, the ocular optical unit 30D guides light from the left eye OBL and the right eye OBR, serving as the object eyes OB, to an image acquisition unit 20E. Specifically, the object eyes OB of the left eye OBL and the right eye OBR are positioned separated by an inter-eye width along the X axis direction, and are both positioned at a common position in the Y axis direction. This means that an illumination unit 40E and the image acquisition unit 20E are arranged at different positions in the X axis direction, corresponding to the positions of the object eyes OB of the left eye OBL and the right eye OBR, and are arranged at a common position in the Y axis direction.

The image acquisition unit 20E includes a left-eye image acquisition unit 20EL for acquiring images of the left eye OBL using light from the left eye OBL reflected by the beam splitter 38 of the ocular optical unit 30D. Moreover, the image acquisition unit 20E includes a right-eye image acquisition unit 20ER for acquiring images of the right eye OBR using light from the right eye OBR reflected by the beam splitter 38 of the ocular optical unit 30D. The left-eye image acquisition unit 20EL and the right-eye image acquisition unit 20ER are configured similarly to the image acquisition unit 20D, and so explanation thereof will be omitted. The left-eye image acquisition unit 20EL is an example of a left-eye acquisition unit of technology disclosed herein, and the right-eye image acquisition unit 20ER is an example of a right-eye acquisition unit of technology disclosed herein.

The illumination unit 40E employs an illumination lens 44D for use with illumination light for each of the left eye OBL and the right eye OBR. The illumination lens 44D may be provided common for both the left eye OBL and the right eye OBR, or may be provided independently for each of the left eye OBL and the right eye OBR. The light source includes a light source 46L for use with the left eye OBL and a light source 46R for use with the right eye OBR. Note that although omitted in the drawings, the polarizing element 45Da and the wave plate 45Db (FIG. 7) described above may be omitted, may be employed common to both the left eye OBL and the right eye OBR, and may be provided independently for each of the left eye OBL and the right eye OBR.

This means that light emitted from the light source 46L for the left eye OBL of the illumination unit 40E is reflected by the beam splitter 38, and the left eye OBL is illuminated thereby. Similarly, light emitted from the light source 40R for the right eye OBR is reflected by the beam splitter 38, and the right eye OBR is illuminated thereby. The light from the light source 46L for the left eye OBL and from the light source 40R for the right eye OBR is reflected toward both eyes of the object eyes OB by reflection by the beam splitter 38, and does not head toward the retroreflection element 34. This means that the illumination unit 40E is able to illuminate both eyes of the object eyes OB without the illumination-light light path for illuminating the object eyes OB being via the retroreflection element 34. This means that the illumination light from the illumination unit 40E does not reach the image acquisition unit 20E directly via the retroreflection element 34. This means that scattering light, generated by the illumination light of the illumination unit 40E being incident to the retroreflection element 34, is not acquired as stray light by the image acquisition unit 20E.

The present exemplary embodiment, is configured so as to enable positional adjustment of at least part of the configuration of the eye imaging device 10E. Specifically, the eye imaging device 10E is installed on a table 60 including a controller 64. A face abutting section 62, which includes a forehead abutting portion and a chin rest portion, for fixing the face of a subject with respect to the eye imaging device 10E, is attached to the table 60. The eye imaging device 10E includes a triaxial moving section 56 configured so as to be able to move the entire eye imaging device 10E in each direction of the X axis direction, Y axis direction, and Z axis direction with respect to the table 60, and in opposite directions thereto. The triaxial moving section 56 is connected to the controller 64, and is configured so as to control movement of the eye imaging device 10E. Note that the controller 64 may be implemented by a general purpose computer including a central processing unit (CPU), and detailed explanation thereof will be omitted because configurations of general purpose computers are known.

The eye imaging device 10E includes a drive unit for driving so as to enable independent adjustment of positions of parts of the respective optical systems responsible for the left eye OBL and the right eye OBR inside the eye imaging device 10E. As an example of such a drive unit, description follows of configuration in the present exemplary embodiment for driving an optical system responsible for one out of the left eye OBL or the right eye OBR so as to enable positional adjustment inside the eye imaging device 10E. Specifically, a case will be described in which the optical system of the left eye OBL is adjusted with respect to the position of the optical system that has been adjusted for the right eye OBR, serving as a reference eye, described later. The eye imaging device 10E includes a tri-axial adjustment unit 52X, serving as a drive unit. With a left-eye illumination unit 40EL and a left-eye image acquisition unit 20EL responsible for the left eye OBL serving as an adjustment target optical system 50L, the tri-axial adjustment unit 52X is configured so as to enable the position of the adjustment target optical system 50L to be adjusted inside the eye imaging device 10E in each of the XYZ axis directions and opposite directions thereto.

Moreover, the eye imaging device 10E includes a focal point adjustment unit for adjusting the focal position for each of the left eye OBL and the right eye OBR. Specifically, a focal point adjustment unit 54L responsible for the left eye OBL and a focal point adjustment unit 54R responsible for the right eye OBR are provided. The focal point adjustment unit 54L responsible for the left eye OBL is configured so as to be able to adjust the adjustment target optical system 50L in position independently to the triaxial moving section 56 and the tri-axial adjustment unit 52X in the left eye OBL depth direction (the Z axis direction and the opposite direction thereto). Moreover, the focal point adjustment unit 54R responsible for the right eye OBR is configured so as to enable an adjustment target optical system 50R, which is configured by a right-eye illumination unit 40ER and the right-eye image acquisition unit 20ER, to be adjusted in position independently to the triaxial moving section 56 in the right eye OBR depth direction. The tri-axial adjustment unit 52X and the focal point adjustment units 54L, 54R are connected to the controller 64, and are configured so as to be individually controlled by the controller 64.

The eye imaging device 10E is able to acquire images of the object eye OB as desired by an observer by, for example, driving the focal point adjustment units 54L, 54R and moving the image acquisition unit 20E to adjust the positions of the object eyes OB. Note that the adjustment target optical system is not limited to one containing an illumination unit and an image acquisition unit, and may be any optical system in which at least a site for acquiring an image is adjustable. For example, it may be an optical system in which only an image acquisition unit is the adjustable target. Namely, it may be an optical system including each of the left-eye illumination unit 40EL and the left-eye image acquisition unit 20EL as adjustable targets.

The triaxial moving section 56, the tri-axial adjustment unit 52X, and the focal point adjustment units 54L, 54R are examples of drive units of the present disclosure. Moreover, the triaxial moving section 56, the tri-axial adjustment unit 52X, and the focal point adjustment units 54L, 54R are examples of position adjustment units of the present disclosure. The controller 64 is an example of a control unit of the present disclosure. Moreover, adjustment of the position of the right eye of the object eyes OB is an example of a function of a right-eye position adjustment unit of the present disclosure, and adjustment of the position of the left eye of the object eyes OB is an example of a function of a left-eye position adjustment unit of the present disclosure. Adjustment of the positions of the right eye and the left eye of the object eyes OB may be performed by at least one out of the focal point adjustment unit or the tri-axial adjustment unit described above, and obviously may be performed by independent configuration.

The eye imaging device 10E is able to adjust the positions of the left eye OBL and the right eye OBR independently due to including the position adjustment unit as described above.

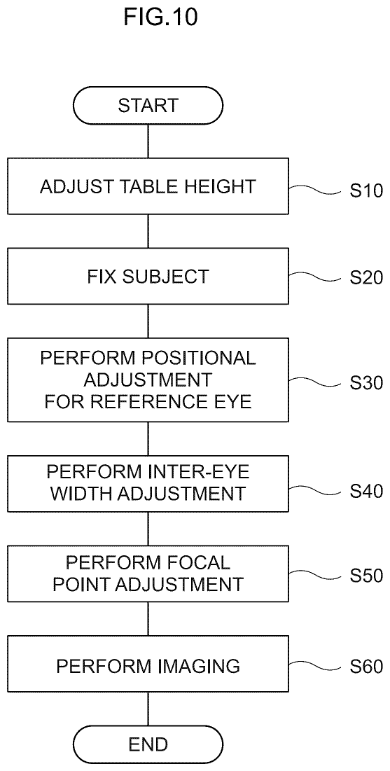

Namely, description follows, with reference to FIG. 10, regarding a flow of processing in the eye imaging device 10E, including processing to adjust the position of both eyes of the left eye OBL and the right eye OBR applied as the object eyes in the eye imaging device 10E. The flow of processing illustrated in FIG. 10 is executed by a controller 64.

Firstly, as rough device positional alignment, at step S10, the position of the eye imaging device 10E, in this case the height of the table 60, is adjusted by operation of an observer such that the position of the eye imaging device 10E corresponds to the position of the face of the subject with the object eye OB, this being such that the forehead abutting portion and the chin rest portion of the face abutting section 62 configured as described above correspond to the position of the face of the subject. Next, at step S20, the eye imaging device 10E notifies the subject with message information or the like to fix the face of the subject to the forehead abutting portion and the chin rest portion of the face abutting section 62 as described above. This thereby enables the face of the subject to be fixed to the eye imaging device 10E. Then at step S20, positional adjustment is performed by operation of the observer while referencing images being acquired with the image acquisition unit 20E, so as to align at least a central position of images and a height position of the object eye OB.

Next, at step S30, the eye imaging device 10E performs positional adjustment for the reference eye. The adjustment of the reference eye is processing to determine one object eye OB as the reference eye from out of the left eye OBL or the right eye OBR, and to adjust the position of the eye imaging device 10E for the determined reference eye. The present exemplary embodiment will be described for a case in which the right eye OBR is determined as the reference eye. At step S30, the position of the eye imaging device 10E is adjusted such that a fundus image of the right eye OBR is at an observable position by operation of the observer while referencing images being acquired with the right-eye image acquisition unit 20ER. Specifically, the relative position between the right eye OBR and the eye imaging device 10E is adjusted such that the fundus image of the reference eye (namely, the right eye OBR) is observable by driving the triaxial moving section 56, and the eye imaging device 10E being moved in the respective XYZ axial directions and the opposite directions thereto.

Next, at step S40, the eye imaging device 10E performs positional adjustment on the other object eye OB (namely, on the left eye OBL) by performing inter-eye width adjustment. The inter-eye width adjustment is processing to configure the device so as to be able to acquire respective images of the left-eye image acquisition unit 20EL and the right-eye image acquisition unit 20ER according to the inter-eye width of the subject. Specifically, at step S40, at least the position of the left-eye optical system in the eye imaging device 10E is adjusted by positional adjustment in the X axis direction such that a fundus image of the left eye OBL is at an observable position, by operation of the observer while referencing images being acquired with the left-eye image acquisition unit 20EL. In the present exemplary embodiment, the relative position between the left eye OBL and the eye imaging device 10E is adjusted such that a fundus image of the adjustment target object eye (i.e. the left eye OBL) is observable by driving the tri-axial adjustment unit 52X, and moving the adjustment target optical system including the left-eye illumination unit 40EL and the left-eye image acquisition unit 20EL in the respective XYZ axial directions and the opposite directions thereto.

Next, in the eye imaging device 10E, at step S50, focal point adjustment is performed for each of the left eye OBL and the right eye OBR by driving the focal point adjustment units 54L, 54R individually under operation of the observer. The eye imaging device 10E is thereby adjusted to a position where the eye imaging device 10E is able to image fundus images of each of the left eye OBL and the right eye OBR.

Next, at step S60, the eye imaging device 10E performs imaging of fundus images in a state in which the positions of each of the left eye OBL and the right eye OBR have been adjusted by operation of the observer.

As described above, in the present exemplary embodiment, even in cases in which application is made to both eyes of the left eye and the right eye as the object eyes, respective images can be acquired for each of the left eye and the right eye, and the illumination light does not become stray light and appear as a ghost image in the images of the left eye and the right eye when each of these respective images is being acquired.

Note that technology disclosed herein is not limited to acquiring respective images of the left eye OBL and the right eye OBR. For example, a configuration may be adopted in which, for the two eyes as object eyes OB, an image of one of the object eyes OB is acquired from out of the left eye OBL and the right eye OBR, and an image of a fixation target or the like is presented to the other of the object eyes OB. In cases in which an image of a fixation target or the like is presented to the other of the object eyes OB, instead of the image sensor 24, a display device may be arranged for displaying the fixation target, or an ophthalmic device may be arranged for emitting light of a fixation target visible by the object eye OB. In such cases, processing for acquiring images and processing for presenting images may be performed, for each of the left eye OBL and the right eye OBR, interchangeably for both eyes by providing a non-illustrated changeover device for driving to enable changeover between the image sensor 24 and a device for presenting images.

Moreover, in the present exemplary embodiment, although description has been given of an example of a configuration including a relay optical system for relaying, to the image sensor 24 (FIG. 7), a position (conjugate position) where light from the object eye OB (from each of the left eye OBL and the right eye OBR) has been retroreflected by the retroreflection element 34 and condensed, technology disclosed herein is not limited thereto. For example, a configuration may be adopted of an image acquisition unit (left-eye image acquisition unit 20EL and right-eye image acquisition unit 20ER) that includes, an image sensor, and an ocular lens for condensing light from the object eye OB (from each of the left eye OBL and the right eye OBR) onto the image sensor.

### Seventh Exemplary Embodiment

Next, description follows regarding a seventh exemplary embodiment. Note that the seventh exemplary embodiment is configured similarly to the exemplary embodiments described above, and so the same reference numerals are appended to the same parts thereof, and detailed explanation thereof will be omitted. Moreover, description follows of parts differing from the exemplary embodiments described above.

In the sixth exemplary embodiment, a case was described in which respective images are acquired for both eyes of the object eyes OB, or an image is acquired from one object eye and an image was presented to the other object eye. The seventh exemplary embodiment is an eye imaging device applied to acquire an image of one of a left eye and a right eye as an object eye OB, while presenting another image to the object eye OB or observing the object eye OB.

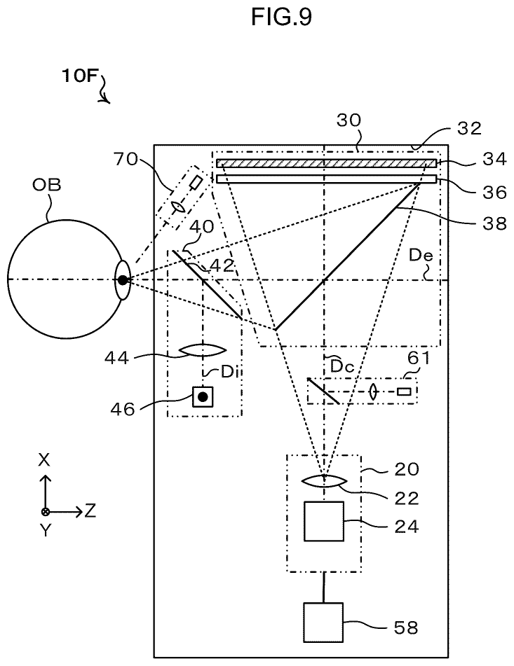

FIG. 9 illustrates an example of a configuration of an eye imaging device 10F according to the present exemplary embodiment.

As illustrated in FIG. 9, the eye imaging device 10F includes an image display unit 61 and a position observation unit 70. The image display unit 61 is a functional unit for presenting images to the object eye OB using an image display. The position observation unit 70 is a functional unit for observing a position of the object eye OB. The image display unit 61 is an example of a projection unit of technology disclosed herein. The position observation unit 70 is an example of an observation unit of the present disclosure.

The image display unit 61 presents an image to the object eye OB by image display of a fixation target or the like. This image may be a structural object observable by the subject, or may be a light point from a point light source as an example of image display of a fixation target or the like. Note that the image display unit 61, serving as a projection unit of the present disclosure, may project an image arranged at a position having a conjugate relationship to a posterior eye portion (namely, the fundus) of the object eye OB by projection onto the posterior eye portion of the object eye OB. The position observation unit 70 may, for example, present an image for an observer to observe a position of the object eye OB by acquiring an image of the pupil Ea of the object eye OB using an imaging device. The position observation unit 70 may include a display device (omitted in the drawings) for displaying an image acquired for positional observation. The display device for displaying the acquired image is an example of a display unit of technology disclosed herein. The location on the object eye OB for checking the position of the object eye OB is not limited to being the pupil Ea, and may be any site on the object eye OB.

As described above, the present exemplary embodiment enables positional alignment of the object eye OB when acquiring images of the object eye OB and checking the state of the object eye OB by acquiring images of the object eye OB, while also providing an image to the object eye or observing the object eye OB.

Moreover, in the present exemplary embodiment, a position adjustment unit 58 may be provided to move the image acquisition unit 20. For example, the observer is able to acquire a desired image of the object eye OB by driving the position adjustment unit 58 according to the position of the pupil of the object eye OB as acquired by the image acquisition unit 20 to move the image acquisition unit 20 so as to adjust a position of the pupil of the object eye OB in the acquired images. The position adjustment unit 58 is an example of a drive unit of technology disclosed herein.

Although explanation of technology disclosed herein has been given using exemplary embodiments, the technical scope of technology disclosed herein is not limited to the scope described in the above exemplary embodiments. Various modifications and improvements may be implemented to the above exemplary embodiments within a range not departing from the spirit thereof, and embodiments including such modifications and improvements are encompassed in the technical scope of technology disclosed herein.

Moreover, processing executed by the above exemplary embodiments may be implemented by a program stored on an auxiliary storage device or the like being executed on a computer. For such processing, processing of at least a part of the program may be implemented by hardware. Furthermore, in order to execute the processing of the exemplary embodiments described above using a computer, a program written with code to enable the above processing to be performed by a computer may be distributed stored on a storage medium or the like, such as an optical disk or the like. The program of the present disclosure may be provided as a program product. Such a program product includes various products for providing programs. For example, the program product encompasses programs for provision over a network such as the Internet or the like, and a non-transitory recording medium, such as a CD-ROM, DVD-ROM, or the like, saved with the program.

All publications, patent applications and technical standards mentioned in the present specification are incorporated by reference in the present specification to the same extent as if each individual publication, patent application, or technical standard was specifically and individually indicated to be incorporated by reference. The entire content of the disclosure of Japanese Patent Application No. 2023-124985 filed on Jul. 31, 2023, is incorporated by reference in the present specification.

1. An eye imaging device for imaging an eye, the eye imaging device comprising:
a retroreflection unit that is configured to retroreflect light from the eye;
a beam splitter that is arranged on a light path of light from the eye to the retroreflection unit; and
an acquisition unit that is configured to acquire, from among the light from the eye, light via the retroreflection unit and the beam splitter.

2. The eye imaging device of claim 1, wherein:
the retroreflection unit, the beam splitter, and the acquisition unit are arranged in a row along a second direction intersecting with a first direction connecting the eye and the beam splitter, and
the acquisition unit is configured to acquire, from among the light from the eye, light that has been retroreflected by the retroreflection unit and transmitted by the beam splitter.

3. An eye imaging device for imaging an eye, the eye imaging device comprising:
a retroreflection unit that is configured to retroreflect light from the eye;
a beam splitter that is configured to reflect, and guide to the retroreflection unit, light from the eye, and to transmit light that has been retroreflected at the retroreflection unit; and
an acquisition unit that is configured to acquire, from among the light from the eye, light that has been retroreflected by the retroreflection unit and transmitted by the beam splitter.

4. The eye imaging device of claim 2, wherein:
the acquisition unit includes an imaging lens and an image sensor, and
the imaging lens is arranged at a position such that a pupil of the imaging lens and a pupil of the eye have a conjugate relationship, and is configured to form an image of a posterior eye portion of the eye as an image on the image sensor.

5. The eye imaging device of claim wherein:
the acquisition unit includes an image sensor, and
the image sensor is arranged at a position having a conjugate relationship with an anterior eye portion of the eye, and is configured to image the anterior eye portion of the eye.

6. The eye imaging device of claim 2, wherein:
the eye imaging device further comprises an illumination unit that includes a light source for emitting light and is configured to illuminate the eye with light from the light source, and
the retroreflection unit and the beam splitter are configured to guide light from the illumination unit that has been reflected by the eye to the acquisition unit.

7. The eye imaging device of claim 6, wherein the illumination unit is configured to illuminate the eye other than via the retroreflection unit.

8. The eye imaging device of claim 6, wherein the illumination unit is arranged at an opposite side of the beam splitter from the eye and is configured to illuminate the eye with light transmitted by the beam splitter.

9. The eye imaging device of claim 6, wherein:
the beam splitter is a polarizing beam splitter, and
the eye imaging device further comprises:
a polarization control unit that is arranged on a light path of light for illuminating the eye by the illumination unit and on the light path between the light source and the polarizing beam splitter; and
a wave plate that is arranged on a light path of light from the eye to the acquisition unit and on the light path between the retroreflection unit and the polarizing beam splitter.

10. The eye imaging device of claim 6, wherein:
the retroreflection unit is configured to retroreflect light from the eye at a first region, and to retroreflect light for illuminating the eye at a second region different from the first region,
the illumination unit includes a separate beam splitter that is arranged on a light path of light for illuminating the eye and on the light path between the light source and the second region, and
the second region is configured to illuminate the eye via the separate beam splitter.

11. The eye imaging device of claim 6, wherein:
the illumination unit includes a separate retroreflection unit for retroreflecting light for illuminating the eye, and a separate beam splitter that is arranged on a light path of light for illuminating the eye and on the light path between the light source and the separate retroreflection unit, and
the separate retroreflection unit is configured to illuminate the eye via the separate beam splitter.

12. The eye imaging device of claim 6, wherein:
the illumination unit includes a separate beam splitter that is arranged on a light path of light from the eye to the beam splitter and on the light path between the eye and the beam splitter, and
the separate beam splitter is configured to reflect light emitted from the light source so as to illuminate the eye, and to transmit light reflected from the eye.

13. The eye imaging device of claim 12, wherein:
the beam splitter is a polarizing beam splitter, and
the eye imaging device further comprises a wave plate that is arranged on a light path of light from the eye to the acquisition unit and on the light path between the polarizing beam splitter and the retroreflection unit.

14. The eye imaging device of claim 1, wherein:
the beam splitter, and the acquisition unit are arranged in a row along a second direction intersecting with a first direction connecting the eye, the beam splitter, and the retroreflection unit, and
the acquisition unit acquires, from among the light from the eye, light that has been retroreflected by the retroreflection unit and reflected by the beam splitter.

15. An eye imaging device for imaging an eye, the eye imaging device comprising:
a retroreflection unit that is configured to retroreflect incident light;
a beam splitter that is configured to transmit, and guide to the retroreflection unit, light from the eye, and to reflect, and guide to an acquisition unit, light that has been retroreflected by the retroreflection unit; and
the acquisition unit, which is configured to acquire, from among the light from the eye, light that has been retroreflected by the retroreflection unit and reflected by the beam splitter.

16. The eye imaging device of claim 14, wherein:
the acquisition unit includes an imaging lens and an image sensor, and
the imaging lens is arranged at a position such that a pupil of the imaging lens and a pupil of the eye have a conjugate relationship, and is configured to form an image of a posterior eye portion of the eye as an image on the image sensor.

17. The eye imaging device of claim 14, wherein:
the acquisition unit includes an image sensor, and
the image sensor is arranged at a position having a conjugate relationship with an anterior eye portion of the eye and is configured so as to image the anterior eye portion of the eye.

18. The eye imaging device of claim 14, further comprising:
an illumination unit that includes a light source for emitting light and that is configured to illuminate the eye with light from the light source,
wherein the retroreflection unit and the beam splitter are configured to guide, to the acquisition unit, light from the illumination unit that has been reflected by the eye.

19. The eye imaging device of claim 18, wherein the illumination unit is configured to illuminate the eye other than via the retroreflection unit.

20. The eye imaging device of claim 18, wherein:
the illumination unit is arranged at an opposite side of the beam splitter from the acquisition unit, and
the beam splitter is configured so as to reflect, and guide to the eye, light from the illumination unit.

21. The eye imaging device of claim 18, wherein:
the beam splitter is a polarizing beam splitter, and
the eye imaging device further comprises:
a polarization control unit that is arranged on a light path of light for illuminating the eye by the illumination unit and on the light path between the light source and the polarizing beam splitter; and
a wave plate that is arranged on a light path of light from the eye to the acquisition unit and on the light path between the retroreflection unit and the polarizing beam splitter.

22. The eye imaging device of claim 18, wherein:
the illumination unit includes a separate beam splitter that is arranged on a light path of light from the eye to the beam splitter and on the light path between the eye and the beam splitter, and
the separate beam splitter is configured to reflect light emitted from the light source so as to illuminate the eye, and to transmit light reflected from the eye.

23. The eye imaging device of claim 22, wherein:
the beam splitter is a polarizing beam splitter, and
the eye imaging device further comprises a wave plate that is arranged on a light path of light from the eye to the acquisition unit and on the light path between the polarizing beam splitter and the retroreflection unit.

24. (canceled)

25. The eye imaging device of claim 1, wherein:
the acquisition unit includes a left-eye acquisition unit for acquiring light from a left eye of the eye and a right-eye acquisition unit for acquiring light from a right eye of the eye, and
the beam splitter and the retroreflection unit are configured to guide light from the right eye to the right-eye acquisition unit and to guide light from the left eye to the left-eye acquisition unit.

26.-31. (canceled)

32. The eye imaging device of claim 1, wherein:
the acquisition unit includes a left-eye acquisition unit for acquiring light from a left eye of the eye and a right-eye acquisition unit for acquiring light from a right eye of the eye, with the beam splitter and the retroreflection unit being configured to guide light from the right eye to the right-eye acquisition unit and to guide light from the left eye to the left-eye acquisition unit, and
the eye imaging device further comprises:
a left-eye position adjustment unit configured to adjust a position of the left-eye acquisition unit with respect to a position of the left eye and a right-eye position adjustment unit configured to adjust a position of the right-eye acquisition unit with respect to a position of the right eye, and
a control unit that is configured to, after adjusting a corresponding position of the left-eye acquisition unit or a corresponding position of the right-eye acquisition unit for one of the left eye or the right eye, then adjust the position of the other thereof.

33. An eye imaging method for an eye imaging device that includes a retroreflection unit for retroreflecting light from an eye and that is configured to image the eye using light that has been retroreflected by the retroreflection unit, the eye imaging method comprising:
guiding light from the eye, which is arranged on a light path of light from the eye to the retroreflection unit, toward the retroreflection unit, and guiding light from the eye, which has been retroreflected at the retroreflection unit, in a different direction from the direction toward the retroreflection unit, and
acquiring light that has been guided in the different direction from the direction toward the retroreflection unit.
